(19) United States
(12) Patent Application Publication
(10) Pub. No.: US 2007/0293842 A1
(43) Pub. Date: Dec. 20, 2007

(54) Cap With Seal Moved By Rotatable Collar And Receptacle With Spreading Surface

(75) Inventors: Barry Knight, Westridge (GB); Peter Bayly, Doreen (AU); Kon Euan Wong, Glen Waverley (AU); Timothy Matthias Morgan, Carlton North (AU)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Acrux DDS Pty Ltd, West Melbourne (AU)

(21) Appl. No.: 11/632,110

(22) PCT Filed: Jul. 14, 2005
(86) PCT No.: PCT/AU05/01033

§ 371 Date: May 24, 2007

Related U.S. Application Data
(60) Provisional application No. 60/587,493, filed on Jul. 14, 2004.

(51) Int. Cl. A61B 19/00

(52) U.S. Cl. 604/403

(57) ABSTRACT

A container includes a receptacle (6) for holding a substance, drug or liquid, and having an opening (7) to the receptacle 6 through which the substance can be dispensed and having a circular and domed spreading surface (15) adjacent the opening for spreading and the accurate delivery of a thin layer of liquid over an area of skin. The container also includes a closure (3) including a cap (4) having a seal or plug (16) for sealing the receptacle (6), and a collar (5) with a cam follower surface (8) rotatable relative to the cap about an axis, whereby rotation of the collar (5) relative to the cap (4) moves the seal in an axial direction away from a seal position.

### FIELD

This invention relates to a container for containing a substance. It will be convenient to hereinafter describe the invention in relation to this particular application, however the invention has other applications for containing substances other than drugs in liquid form.

### BACKGROUND

It is generally desirable for containers containing a substance to have a closure that closes an opening of the container, whereby the closure can be removed at the discretion of an end user to permit the dispensing of the substance from the container. Where the substance is a liquid it is preferable for the closure to seal the opening to avoid the liquid egressing from the container via the opening. This can be particularly important where the substance is a drug in liquid form as the container may contain a single dose or a set number of doses that must all be taken for the drug to be effective. Furthermore where the drug is in a volatile liquid such as ethanol, the liquid may evaporate via the seal unless the seal is a hermetic seal.

Container closures of the kind such as screw caps, or snap and friction fit plugs that have a seal generally require a level of force to achieve that seal. With closures of the foregoing kind there tends to be a relationship between the level of force and the integrity of the seal so that, for example, a hermetic seal is only achieved using a relatively large force. It should be appreciated that the supplier of the container or the supplier of the substance to be contained by the container will normally prefer to use sufficient force to avoid unintentional or unauthorized removal of the closure. By contrast the end user will normally prefer to remove the closure using minimal force. Previously this has required a compromise between the preferences of the suppliers and the end users.

It would be preferable to provide a container having a closure that addressed some of these problems. In particular it would be desirable for the container to address the issue of the compromise between the preferences of the suppliers and the end users. It would also be preferable for the container to be relatively easy to use by not only the authorized end user but also the supplier of the substance that may be filling the container.

### SUMMARY

According to this invention there is provided a container for containing a substance, the container including:

a receptacle for holding the substance the receptacle having an opening through which the substance can be dispensed;

a closure including a cap having a seal for sealing the opening of the receptacle when the seal is in a seal position, a collar being rotatable relative to the cap about an axis, whereby rotation of the collar relative to the cap moves the seal in the axial direction away from the seal position.

The seal may take any form however it is preferred that the seal be in the form of a plug or spigot. The plug preferably engages an inner surface of the receptacle when in the seal position. The plug may engage the receptacle in a friction fit however this is not essential as, for example, the plug may engage the receptacle in a snap fit.

The plug is preferably moved from the seal position in a linear motion, particularly linear motion that is not rotational. This form of motion is considered less likely to detach or shear the plug from the rest of the cap than if it was moved using for example a rotational motion. The closure and the receptacle may be configured in any suitable manner to limit the motion to linear motion. The preferred configuration is for the cap to include a skirt having at least one rib formed on an inner surface of the skirt. The receptacle forming part of a body with the rib of the skirt being located between splines formed on the body, when the plug is in the seal position. The at least one rib and splines extend in the axial direction which limits movement of the plug to the axial direction. Clearly other configurations may be suitable.

The closure is preferably formed with a cam means to move the cap including the seal from the seal position. The cam means may be achieved by any suitable configuration. The preferred configuration is for the cap to include a cam surface formed thereon for engagement by a follower surface of the collar. The collar also preferably includes a bearing surface which bears against a shoulder of the body so that rotation of the collar results in movement of the cap in the axial direction. Clearly other configurations could be suitable, for example the cam surface and the follower surface may be transposed. Alternatively the bearing surface and the cam surface may be transposed. It is preferred that at least 90 degrees rotation of the collar be sufficient to move the seal from the seal position however other amounts of rotation may also be suitable. The collar may be rotated through 180, 360 or 720 degrees to use the cam means to greater effect.

The closure is preferably configured to limit the amount of movement in the axial direction of the cap relative to the collar. This may be achieved in any suitable manner. It is preferred however that the collar be formed with a lip that can be engaged by one or more stops formed on the cap when the cap reaches a maximum extension position. It is preferred that the maximum extension position be reached by rotation of the collar, however this is not essential.

According to another aspect of this invention there is provided a container for containing a liquid to be spread over a treatment site of the user, the container including:

a receptacle for holding the liquid, the receptacle having an opening through which the liquid can be dispensed, the body having a spreading surface formed adjacent the opening for spreading the liquid on the treatment site of the user.

The spreading surface is preferably domed having a radius of curvature within the range of 90% to 100% of the diameter of the spreading surface. The spreading surface is preferably circular in plan however this is not essential, as for example the spreading surface may be oval in plan. In either case it is preferred that the perimeter of the circle or oval be provided with a curved edge with a radius within the range of 10% to 15% of the diameter of the spreading surface.

The area of the spreading surface is preferably determined having regard to the volume of drug to be dispensed, the viscosity of the drug and the area of the treatment surface over which the drug is to be spread. Where the volume of the drug is within the range of 50 pμm to 500 pμm, the viscosity is approximately 2.5 centipoises at an ambient temperature of 20°, and the treatment site is of 15 cm² to 200 cm², the preferred area of the spreading surface is 1.3 cm².

The opening is preferably determined having regard to the viscosity of the liquid. Where the viscosity of the liquid is 2.5 centipoises at an ambient temperature of 20°, an opening of 4 mm in diameter is preferred.

The invention will be hereinafter described with reference to the attached illustrations that show one example embodiment of a container to which the invention applies. The particularity of the drawings and the following description is not to be considered as limiting the generality of the preceding general definition of the invention.

### BRIEF DESCRIPTION OF FIGURES

FIG. 1 illustrates in isometric view a preferred embodiment of the container to which the invention applies with the closure on the body.

FIG. 2 illustrates in isometric view the container from FIG. 1 with the closure removed from the body and in exploded form.

FIG. 3 illustrates in isometric view the container from FIG. 1 with the assembled closure removed from the body.

FIG. 4 illustrates an elevation view of the body in isolation from the closure from FIG. 1.

FIG. 5 illustrates in cross section the container body through section A-A from FIG. 4.

FIG. 6 illustrates an elevation view of the container from FIG. 1.

FIG. 7 illustrates in cross section the container through section A-A from FIG. 6.

FIG. 8 illustrates an elevation view of the container from FIG. 6 rotated through 90 degrees.

FIG. 9 illustrates in cross section the container through section B-B from FIG. 8.

FIG. 10 illustrates an elevation view of the container from FIG. 6 with the collar rotated through 90 degrees.

FIG. 11 illustrates in cross section the container through section B-B from FIG. 10.

FIG. 12 illustrates an elevation view of the container from FIG. 10 rotated through 90 degrees.

FIG. 13 illustrates in cross section the container through section A-A from FIG. 12.

FIG. 14 illustrates in isometric view an alternate embodiment of the closure.

FIG. 15 illustrates in cross section the closure from FIG. 14.

### DETAILED DESCRIPTION

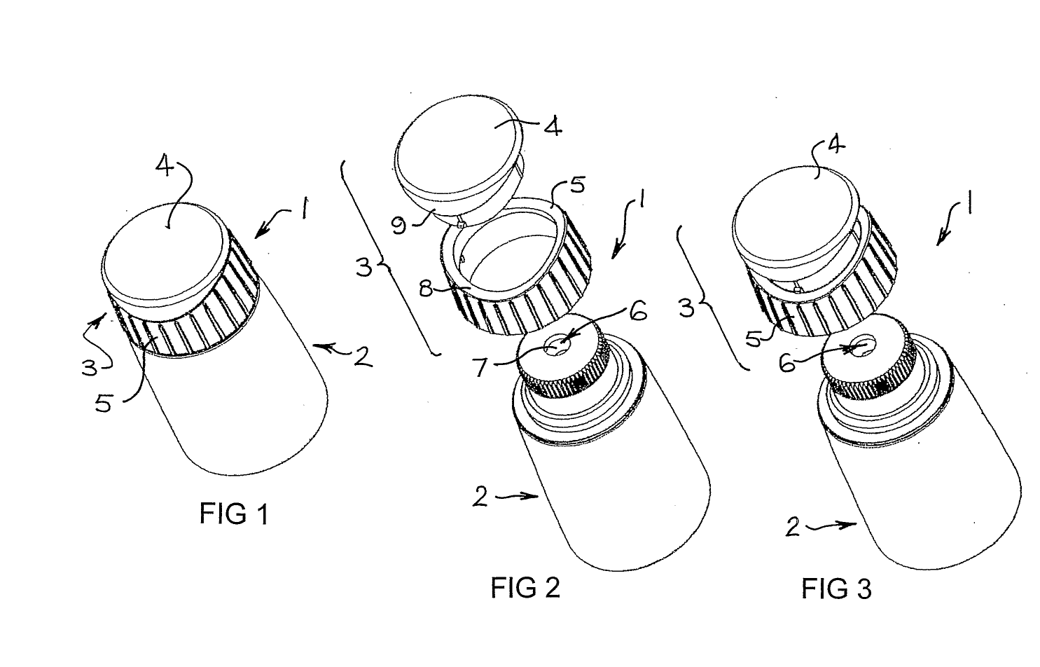

Referring firstly to FIG. 1 which illustrates a preferred embodiment of the container 1. The container 1 illustrated includes, in summary, a body 2 and a closure 3. The closure 3 includes a cap 4 having a seal (not shown in FIG. 1) and a collar 5 surrounding the cap 4 the features of which can be better described with reference to later illustrations.

Referring now to FIG. 2, which shows the container 1, and in particular the closure 2 in an exploded form. It should be noted that it is not intended that the cap 4 be detached from the collar 5. Instead the closure 3 illustrated is configured to limit movement of the cap 4 relative to the collar 5. This configuration will be explained in greater detail with reference to later illustrations.

FIG. 2 shows the closure 3 removed from the body 2 of the container 1 to expose a receptacle 6 or more specifically an opening 7 to the receptacle 6. The receptacle 6 is provided for holding the substance (not shown) and in particular liquids. The container 1 illustrated has an application containing drugs in liquid form, which can be dispensed from the receptacle 6 when the closure 3 is removed from the body 2. The process of removing the closure 3 from the body 2 will be described with reference to later illustrations.

Referring still to FIG. 2, it is convenient to use the exploded form of the container 1 to note the collar 5 has a follower surface 8, which engages a cam surface 9 of the cap 4. The cam and follower surfaces 8, 9 form part of a cam means the function of which will be described with reference to later illustrations.

FIG. 3 is an illustration of how the closure 3 should appear when removed from the body 2, more specifically with the cap 4 captured by the collar 5. In this manner the collar 5 is removed from the body 2 with the cap 4 thus avoiding the collar 5 interfering with dispensing of the substance from the receptacle 6.

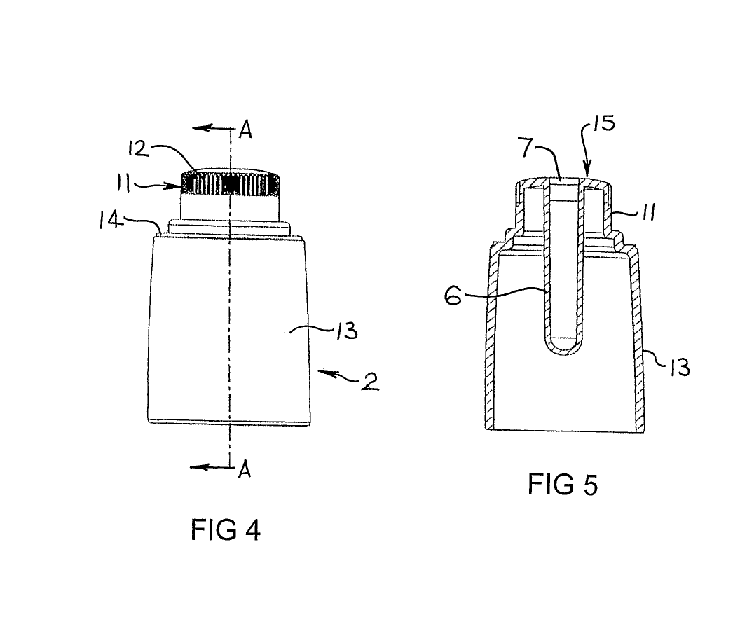

Referring now to FIG. 4, which illustrates the body 2 in isolation from the closure 3. The body 2 has an upper wall portion 11, which is formed with a splined cylindrical surface 12 extending axially of the body 2. The function of the spline 12 will be explained with reference to later illustrations. The body 2 also has a lower wall portion 13 that is principally provided for facilitating grasping of the body 2 by an end user. A shoulder 14 is provided between the upper and lower wall portions 11, 13 the function of which will be explained with reference to later illustrations.

Referring now to FIG. 5, which shows the receptacle 6 relative to the upper and lower wall portions 11, 13. The size of the receptacle 6 is principally determined by the volume of substance it is required to hold. The application of the container 1 illustrated is to contain a single dose of a drug in a relatively small volume. Accordingly the size of the receptacle 6 may vary according to the application of the container 1. The variation may be to the extent that the lower wall portion 13 is not required as the end user can effectively grasp the receptacle 6 itself.

The receptacle 6 as shown in FIG. 5 is formed integrally with the rest of the body 1. It can be envisaged that the receptacle 6 may have or be lined with an insert (not shown) located therein for retaining the liquid or may be a separate component assembled to the body. This may be appropriate where the body 2, including the receptacle, is formed from a material that would react with the liquid. An insert or separate receptacle made from a material that is not reactive to the liquid may be used.

The size of the opening 7 to the receptacle 6 in the embodiment illustrated in at least FIG. 5 is related to the viscosity of the drug. This is so that the liquid can be retained within the receptacle 6 by surface tension even after the closure 3 has been removed from the body 2 and the receptacle 6 inverted with the opening pointing downwards towards the treatment site (skin). Once the upper surface 15 of the body 2 makes contact with the skin of the end user the surface tension is broken allowing the liquid to be dispensed from the receptacle 6. Once contact of the upper surface 15 with skin is broken, the flow of fluid will cease due to the surface tension of the fluid. Accordingly the size of the opening 7 may vary according to the viscosity of the liquid. In the proposed application for this container 1 the drug in liquid form has a viscosity of 2.5 centipoises at an ambient temperature of 20°, whereby an opening 7 of 4.0 mm in diameter has been found to be suitable.

Referring still to FIG. 5 it can be noted that the body 2 has a domed upper surface 15. The application of this container 1 is for a liquid that is to be applied to the skin of the end user with this surface 15 facilitating spreading the drug over the skin in a controlled thin layer. The spreading surface 15 illustrated is of slightly convex form with radiused edges and circular shape. The shape may vary and be for example oval. The domed or convex nature of the spreading surface 15 facilitates a positive connection with the skin of the end user.

The diameter of the spreading surface 15 is determined by having regard to the volume of the drug to be dispensed, the viscosity of the drug and the area of the skin surface over which the drug is to be spread.

The receptacle 6 preferably holds between 50 μl to 500 μl of liquid having a viscosity of approximately 2.5 centipoises at an ambient temp of 20°.

A domed upper surface 15 of 13.0 mm diameter has been found to provide a suitable surface for applying an accurate and reproducible amount of liquid per unit area of the skin over an area of skin of 15 cm² to 200 cm²

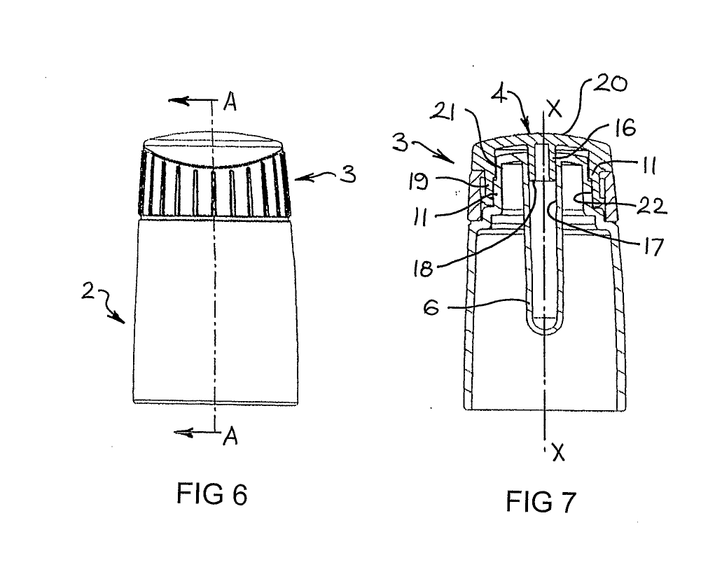

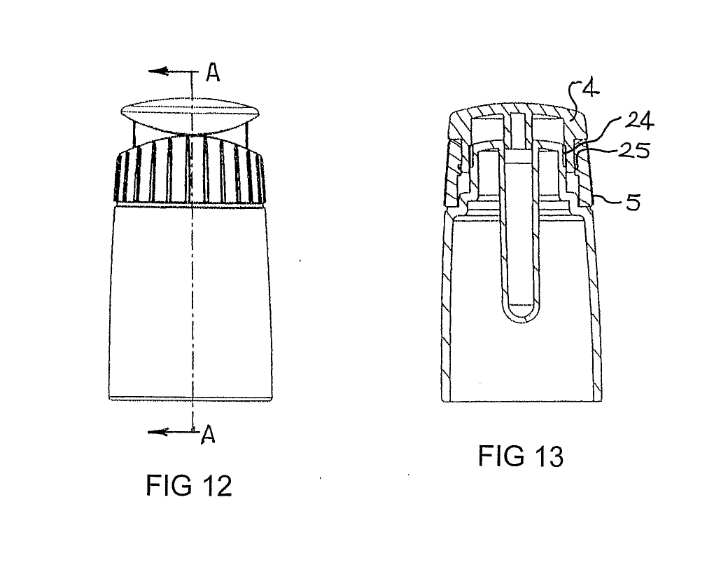

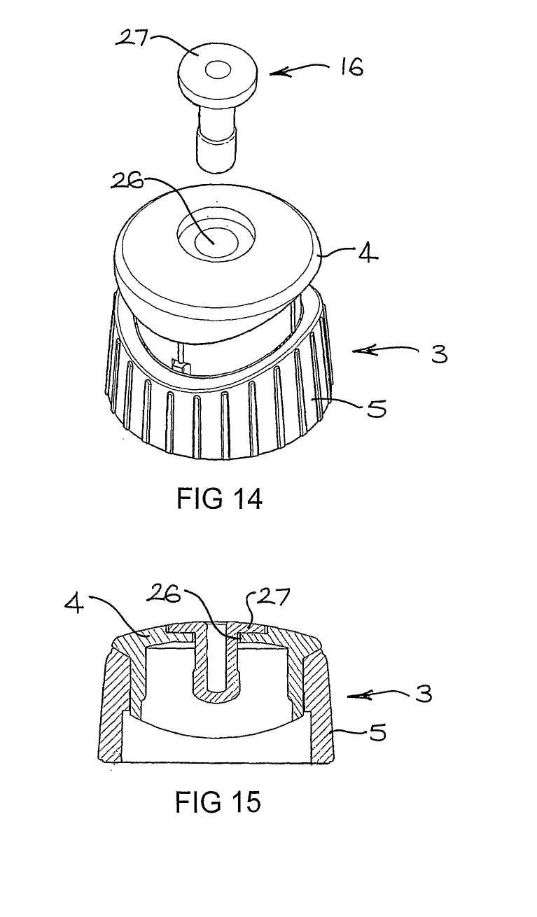

The illustration of the container in FIG. 6 has the closure 3 on the body 2, and is provided to appreciate the sectional illustration in FIG. 7. FIG. 7 illustrates the closure 3 including a preferred embodiment of a seal 16, which in reference to the illustration can be described as a plug or a spigot. The plug 16 is illustrated in FIGS. 6 to 9 in a seal position with the plug 16 engaging the receptacle 6, and in particular an inner surface 17 of the receptacle 6. The plug 16 preferably engages the inner surface 7 of the receptacle 6 in a friction fit. A free end 18 of the plug 16 illustrated in at least FIG. 7 is bulged to achieve the friction fit. The plug 16 illustrated in FIGS. 6 to 13 is formed integrally with the cap 4. The plug 16 may not necessarily be formed integrally with the cap 4 but rather formed separately as illustrated in FIGS. 14,15.

The closure 3 illustrated in FIG. 7 also includes a skirt 19 extending from a top 20 of the cap 4, which surrounds the plug 16. The skirt 19 engages the upper wall portion 11 of the body 2 in a manner that limits movement of the plug 16, and the cap 4 to the direction of the axis x-x. This is achieved by the skirt 19 including at least one rib 21 formed on opposing sides of the inner surface 22 of the skirt 19 that is/are located within the splines 12 formed on the upper wall 11 of the body 2. The splines 12 and ribs 21 extending in the direction of the axis x-x limit movement of the cap 4 to the direction of the axis x-x.

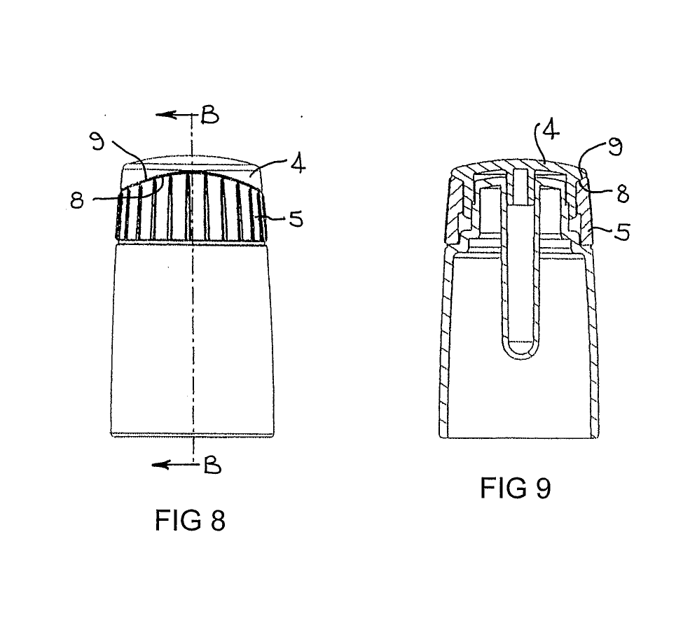

The illustration of the container in FIGS. 8 and 9 show in elevation the cam surface 9 and the follower surface 8 of the cap 4 and collar 5 respectively.

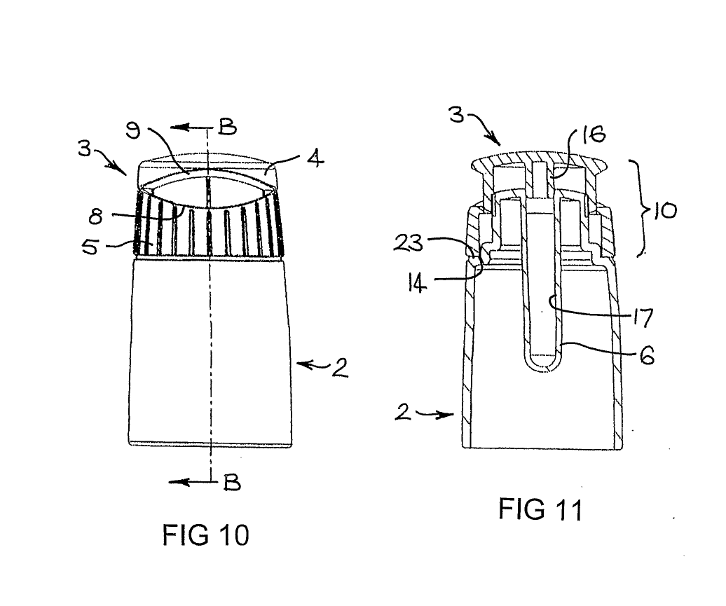

Referring now generally to FIGS. 10 to 13, which illustrates the collar 5 rotated through 90 degrees from the arrangement illustrated in FIGS. 6 to 9. The rotation of the collar 5 causes the cam means 10 to take effect. The cam means 10 also includes a bearing surface 23 formed on an end of the collar opposing the follower surface 8. The bearing surface engages the shoulder 14 of the body 2 when the closure 3 is on the body 2. This arrangement of the cam means 10 causes the cap 4 including the plug 16 to move axially away from a seal position when the collar 5 is rotated. This is due to the realignment of the cam and follower surfaces 9, 8 of the cap 4 and collar 5 respectively so that peaks of those surfaces are aligned. This substantially disengages the plug 16, or at least the bulge of the plug from its friction fit engagement with the inner surface 17 of the receptacle 6 allowing the closure 3 to be detached from the body 2.

Referring in particular to FIG. 13 that illustrate stops 24 formed on the cap 4 that engage a lip 25 of the collar 5 in this position to limit the extent of the axial movement of the cap 4.

Referring now to FIGS. 14 and 15 which illustrate an alternate preferred embodiment of the plug 16 and cap 4. The top 20 of the cap 4 is formed with an opening 26 to receive the plug 16. The plug 16 includes a head portion 27 that limits the passage of the plug 16 through the opening 26. This arrangement allows the receptacle 6 to be filled with substance with the closure 3 in place, while still permitting the closure 3 to be subsequently plugged with plug 16.

It should be appreciated that this arrangement allows the closure to be fitted by axially depressing the cap onto the body. The friction fit produced when the cap is fitted can be such that the end user can't move the closure axially relative to the body. Instead the closure can only be removed by using the mechanical benefit of using the cam means by rotating collar to overcome the friction fit.

Furthermore the configuration of the spreading surface permits the user to spread the liquid over the treatment site in a controlled thin layer without the liquid making contact with other parts of the user.

Finally it is to be understood that various alterations and or additions may be introduced without departing from the spirit or ambit of the invention as provided in the general definition of the invention.

1-15. (canceled)

16. A container for containing a substance to be spread on a treatment site of a user, the container including:
a body having a receptacle for holding the substance the receptacle having an opening through which the substance can be dispensed; the body having a spreading surface formed adjacent the opening for spreading the substance on the treatment site of the user.
a closure including a cap having a seal for sealing the opening of the receptacle when the seal is in a seal position, a collar being rotatable relative to the cap about an axis, whereby rotation of the collar relative to the cap moves the seal in the axial direction away from the seal position.

17. A container according to claim 16 wherein the seal is in the form of a plug having a free end that engages an inner surface of the receptacle when the seal is in the seal position.

18. A container according to claim 17 when the plug is formed integrally with the cap.

19. A container according to claim 17 wherein the plug is formed separately from the cap.

20. A container according to claim 19 wherein the cap is formed with an opening to receive the plug, the plug having a head portion formed remote from the free end that is sized to limit movement of the plug through the opening.

21. A container according to claim 17 wherein the free end of the plug engages the inner surface of the receptacle in a friction fit.

22. A container according to claim 16 wherein the cap includes a skirt having at least one rib formed on an inner surface of the skirt, the receptacle forming part of a body having splines formed on an outer surface thereof, whereby the rib and splines extend in the axial direction with the rib located between the splines in the seal position so as to limit movement of the plug away from the seal position to the axial direction.

23. A container according to claim 22 wherein each spline is spaced from each adjacent spline by a spacing which is substantially similar to a thickness dimension of each rib.

24. A container according to claim 23 wherein the closure includes cam means associated with the cap and collar to move the seal from the seal position.

25. A container according to claim 24 wherein the cam means includes a cam surface formed on the cap for engagement by a follower surface formed on the collar, the collar also having a bearing surface which bears against a shoulder of the body so that rotation of the collar causes movement of the seal from the seal position.

26. A container according to claim 25 wherein the collar is rotated through any one of 90°, 180°, 360°, or 720° to move the seal from the seal position.

27. A container according to claim 16 wherein the collar is formed with a lip that is engaged by at least one stop formed on the cap when the cap reaches a maximum extension position.

28. A container according to claim 16, wherein the spreading surface is circular and domed having a radius of curvature within the range of 90% to 100% of the diameter of the spreading surface.

29. A container according to claim 16, wherein the spreading surface has a curved edge with a radius of curvature within the range of 10% to 15% of the diameter of the spreading surface.

30. A container according to claim 16 wherein the substance is a transdermal drug in liquid form and the treatment site is skin.
